(19) United States
(12) Patent Application Publication
(10) Pub. No.: US 2022/0251512 A1
(43) Pub. Date: Aug. 11, 2022

(54) SERUM-FREE POLYPEPTIDE COMPOSITION FOR PROMOTING PROLIFERATION OF MESENCHYMAL STEM CELLS

(71) Applicant: GUANGZHOU SALIAI STEM CELL SCIENCE AND TECHNOLOGY CO., LTD., Guangzhou, Guangdong (CN)

(72) Inventors: Haijia CHEN, Guangzhou, Guangdong (CN); Xiaohu GE, Guangzhou, Guangdong (CN); Xiaoyan WANG, Guangzhou, Guangdong (CN); Kangyi QI, Guangzhou, Guangdong (CN); Jiaohua JIANG, Guangzhou, Guangdong (CN)

(73) Assignee: GUANGZHOU SALIAI STEM CELL SCIENCE AND TECHNOLOGY CO., LTD., Guangzhou, Guangdong (CN)

(21) Appl. No.: 17/613,941

(22) PCT Filed: Dec. 25, 2019
(86) PCT No.: PCT/CN2019/128193

§ 371 Date: Nov. 23, 2021

(30) Foreign Application Priority Data
May 23, 2019 (CN) 201910432549.0

(51) Int. Cl. C12N 5/0775

(52) U.S. Cl. CPC C12N 5/0662; C12N 2500/25; C12N 2500/90; C12N 2500/36; C12N 2501/11; C12N 2500/32

(57) ABSTRACT

The present invention provides a serum-free polypeptide composition for promoting proliferation of mesenchymal stem cells. The composition mainly comprises: 10-100 μg/L of tripeptide-1; 1-20 μg/L of tripeptide-2, 1-20 μg/L of hexapeptide-9, 1-20 μg/L of palmitoyl hexapeptide-12; and 10-100 μg/L of a laminin-derived peptide. The serum-free polypeptide composition provided in the present disclosure has clear chemical components without animal origins or serum, can achieve rapid proliferation of the mesenchymal stem cells, and maintains the biological characteristics and immunophenotypic stability of the mesenchymal stem cells while solving the problem of the insufficient quantity of cells.

### CROSS REFERENCE TO RELATED APPLICATIONS

This application claims priority to Chinese Patent Application No. 201910432549.0, entitled “SERUM-FREE POLYPEPTIDE COMPOSITION FOR PROMOTING PROLIFERATION OF MESENCHYMAL STEM CELLS,” filed May 23, 2019, the entire contents of which are incorporated herein by reference.

### FIELD

The present disclosure relates to the technical field of cell proliferation, and in particular, to a serum-free polypeptide composition for promoting proliferation of mesenchymal stem cells.

### BACKGROUND

Mesenchymal stem cells (MSCs) are derived from early development mesoderm, and are non-hematopoietic stem cells, which are widely present in bone marrow, subcutaneous fat, bone outer membrane, muscle, synovium, synovia, liver, peripheral tissue, umbilical cord, umbilical cord blood, placenta and other tissues. MSCs have a highly self-updating capability and a multidirectional differentiation potential, can be cultured in vitro; MSCs not only can support the growth of hematopoietic stem cells, but also has an immune regulation effect. Under different induction conditions, MSCs can be differentiated into bone, cartilage, muscle, nerve, myocardium, endothelial and fat, etc. in vitro. MSCs still possess multi-directional differentiation potential after continuous passage culture and cryopreservation, and can be used as an ideal seed cell to repair tissue and organ damage caused by aging and pathological changes. Therefore, MSCs have a wide clinical application prospect, and are preferred seed cells for cell replacement therapy and tissue engineering, and are research hotspots in the field of transplantation and autoimmune disease treatment.

Most of the medium used in the existing mesenchymal stem cell culture methods contain animal serum, such as fetal bovine serum (FBS), which is the most common serum. FBS has a complex composition and contains heterogeneous proteins, and is easy to carry viruses or infected by mycoplasma. On the other hand, FBS varies greatly from batch to batch and the source is unstable, which has a greater impact on the process of large-scale expansion of MSCs in vitro. Current studies have shown that MSCs will phagocytose proteins in the medium during the culture process and the bovine serum albumin contained in the medium can cause the recipient's body to produce anti-bovine serum albumin antibodies to cause an immune response, which leads to failure of the treatment, especially after repeated injection of MSCs. Therefore, researchers began to develop alternatives to FBS.

There are many types of serum substitutes on the market, but most of them still contain some animal-derived ingredients, such as Ultraser G (Pall BioSepra). Some serum substitutes are from human serum derivatives, including human serum, platelet derivatives, and umbilical cord serum. Although these products are human-derived, their ingredients are still unclear, and the resources are few, mass production is difficult to achieve, and it is impossible to guarantee large-scale cultivation of MSCs in vitro.

Serum-free medium (Serum-Free Medium, SFM), as the name implies, does not require addition of animal or human serum in cell culture. However, in order to meet the requirements of cell growth, materials that can function as serum are usually added to culture medium, mainly including several categories such as binding proteins, growth factors, adhesion factors, hormones, and trace elements. The research and development of SFM applied to mesenchymal stem cells has gone through four major stages. The first generation is a serum-free medium in the general sense which uses a variety of biological materials that can replace the function of serum. It contains a large number of animal and plant-derived proteins and unknown components, such as animal or human platelet lysates. Its advantages are higher experimental accuracy, higher repeatability, and higher stability compared with the serum-containing medium. However, due to the unclear chemical composition of the added materials, most of them contain a large amount of animal-derived proteins, which is not conducive to the separation and purification of target protein, and the cost is high. The second generation is a serum-free and animal-derived protein-free medium, wherein the added components do not use animal-derived proteins at all. Instead, various recombinant proteins or animal and plant protein hydrolysates are used. Its advantage is that the stability is improved and the cost is reduced compared with the first generation. The cost is reduced, and the effect is correspondingly improved. However, due to the high cost, it is only suitable for scientific research users with low demand, and it is difficult to be used by enterprises that carry out large-scale production. The third generation is a medium with defined compositions, also known as a double-free medium. The compositions added to the medium are completely serum-free, protein-free or with very low protein content, and the proteins are known. The third-generation SFM is currently the main product on the market. It has obvious advantages: cell culture and production are easy to maintain, and the target protein is easier to be separated and purified, and the cost is greatly reduced. However, at present, one disadvantage of such products is that, the cells cultured in the medium do not have sufficient cell passage ability, which is also the bottleneck that SFM R&D companies urgently need to break through. In addition, the third-generation SFM has high specificity for cultured cells, so only suitable for a few cell lines, and product development is extremely difficult. The fourth generation is a medium with defined chemicals. The chemicals added to the medium do not contain serum and any protein. The unstable protein substances are mainly replaced by compounds. It can be sterilized at high temperature. It is an all-round medium suitable for the growth of a variety of different cells. Currently, there is no such product on the market, and it is still in the research and development stage.

With the rapid development of the industry, there are more and more serum-free culture medium products, and the ones with better reputation and more use are basically produced by STEMPRO® hMSC SFM of GIBCO, MesenCult-XF Medium of STEMCELL Technologies, and so on. These medium are not only expensive and cannot meet the requirements of mass production, but also need to coat the culture container with gelatin when culturing MSCs, which has a high risk of introducing animal-derived proteins.

Polypeptide is a compound formed by linking ct-amino acids together by peptide bonds, which is an intermediate product of protein hydrolysis. Compounds formed by the dehydration and condensation of two amino acid molecules are called dipeptides. Analogously, there are tripeptides, tetrapeptides, pentapeptides, etc. Generally, compounds formed by dehydration and condensation of three or more amino acid molecules can be called polypeptides. Bioactive peptides have a variety of physiological functions, such as hormonal effects, immune regulation, anti-thrombosis, anti-hypertension, cholesterol-lowering, antibacterial, anti-viral, and anti-cancer effects. Peptides are currently widely used in skin care products, with functions such as anti-aging, whitening, and skin repair, and so on.

### SUMMARY

The present disclosure aims to provide a serum-free polypeptide composition, which can effectively promote the rapid growth of mesenchymal stem cells.

In view of this, the present disclosure provides a serum-free polypeptide composition for promoting proliferation of mesenchymal stem cells, comprising:

| | | |
|---|---|---|
| tripeptide-1 | 1~20 | μg/L; |
| tripeptide-2 | 1~20 | μg/L; |
| hexapeptide-9 | 1~20 | μg/L; |
| palmitoyl hexapeptide-12 | 1~20 | μg/L; |
| laminin-derived peptide | 10~100 | μg/L; |
| non-essential amino acids | 1~5 | vol %; |
| glutamine | 1~4 | mmol/L; |
| lipid mixture | 1~5 | vol %; |
| ITS (Insulin, Transferrin, Selenium) | 1~5 | vol %; |
| recombinant human serum albumin | 1~5 | g/L; |
| recombinant human epidermal growth factor | 5~25 | μg/L; |
| recombinant human fibronectin | 5~15 | μg/L; |
| L-glutathione | 1~5 | mg/L; |
| β-mercaptoethanol | 1~5 | mg/L; and |
| α-MEM basal medium | 10.2 | g/L. |

Preferably, content of the tripeptide-1 is 3-18 μg/L.

Preferably, content of the tripeptide-2 is 3-18 μg/L.

Preferably, content of the hexapeptide-9 is 4-18 μg/L.

Preferably, content of the palmitoyl hexapeptide-12 is 3-17 μg/L.

Preferably, content of the laminin-derived peptide is 30-65 μg/L.

Preferably, content of tripeptide-1 is 10 μg/L, content of tripeptide-2 is 10 μg/L, content of hexapeptide-9 is 10 μg/L, content of the palmitoyl hexapeptide is 10 μg/L, and content of the laminin-derived peptide is 60 μg/L.

Preferably, content of the non-essential amino acid is 1 vol %, content of the glutamine is 1-4 mmol/L, content of the lipid mixture is 1 vol %, content of the ITS is 1 vol %, content of the recombinant human serum albumin is 2.5 g/L, content of the recombinant human epidermal growth factor is 20 μg/L, content of the recombinant human fibronectin is 10 μg/L, content of the L-glutathione is 2 mg/L, and content of the β-mercaptoethanol is 2 mg/L.

The application provides a polypeptide composition, which comprises a basal serum-free medium composed of a mixture of non-essential amino acids, glutamine, lipid mixture, ITS, recombinant human serum albumin, recombinant human epidermal growth factor, recombinant human fibronectin, L-glutathione, β-mercaptoethanol and α-MEM basal medium, and further comprises a polypeptide composition composed of tripeptide-1, tripeptide-2, hexapeptide-9, palmitoyl hexapeptide-12 and laminin-derived peptide. The above composition is supplemented with a combination of polypeptides in the basal medium, and the polypeptides have the function of promoting adhesion of collagen and fibronectin, and effectively promoting the synthesis of macromolecules such as cell elastin, collagen, laminin and integrin, promotes cell proliferation and migration. Therefore, the peptide compositions are adopted as a part of the culture medium. The medium has definite chemical components, no animal origin, and no serum. It can realize rapid proliferation of mesenchymal stem cells so that solve the problem of insufficient cell numbers, while maintaining biological characteristics and immunophenotypic stability of the mesenchyme stem cell.

### BRIEF DESCRIPTION OF DRAWINGS

FIG. 1 is a morphological images (40×) of UC-MSCs in the experiment group and the control group of the present disclosure;

FIG. 2 is a graph of the growth curve of UC-MSCs in the experiment groups and the control groups of the present disclosure;

FIG. 3 is a diagram of the adipogenic differentiation results of UC-MSCs in the experiment groups and the control groups of the present disclosure (400×); and

FIG. 4 is a diagram of the osteogenic differentiation results of UC-MSCs in the experiment groups and the control groups of the present disclosure (40×).

### DETAILED DESCRIPTION

In order to make the technical solutions of the present disclosure more clearly understood, the preferred embodiments of the present disclosure will be described below in conjunction with examples. It should be understood that these descriptions are only for further illustrating the features and advantages of the present disclosure, rather than limiting the claims of the present disclosure.

Aiming at the serum-free medium required for mesenchymal stem cells in the prior art, this application provides a polypeptide composition, which can promote the rapid proliferation of mesenchymal stem cells as a serum-free medium. Specifically, an embodiment of the present disclosure discloses a serum-free polypeptide composition for promoting the proliferation of mesenchymal stem cells, comprising:

| | | |
|---|---|---|
| tripeptide-1 | 1~20 | μg/L; |
| tripeptide-2 | 1~20 | μg/L; |
| hexapeptide-9 | 1~20 | μg/L; |
| palmitoyl hexapeptide-12 | 1~20 | μg/L; |
| laminin-derived peptide | 10~100 | μg/L; |
| non-essential amino acids | 1~5 | vol %; |
| glutamine | 1~4 | mmol/L; |
| lipid mixture | 1~5 | vol %; |
| ITS | 1~5 | vol %; |
| recombinant human serum albumin | 1~5 | g/L; |
| recombinant human epidermal growth factor | 5~25 | μg/L; |
| recombinant human fibronectin | 5~15 | μg/L; |
| L-glutathione | 1~5 | mg/L; |
| β-mercaptoethanol | 1~5 | mg/L; |
| α-MEM basal medium | 10.2 | g/L. |

In the above medium, tripeptide-1, tripeptide-2, hexapeptide-9, palmitoyl hexapeptide-12 and laminin-derived peptides are used as a polypeptide combination of the polypeptide composition. Among them, tripeptide-1 can promote the production of elastin and collagen. The α-MEM basal medium is used as a constant volume substrate, and content of tripeptide-1 is 1-20 μg/L; in a specific embodiment, content of the tripeptide-1 is 3-18 μg/L.

The tripeptide-2 is an active tripeptide derived from an elastase inhibitor, which can reduce the synthesis of progerin, and its content is 1-20 μg/L. In a specific embodiment, content of the tripeptide-2 is 3˜18 μg/L.

The hexapeptide-9 is synthesized from six amino acids and is a very stable collagen peptide, which can promote the production of cellular collagen, laminin and integrin; its content is 1-20 μg/L. In a specific embodiment, content of the hexapeptide-9 is 4-18 μg/L.

The palmitoyl hexapeptide-12 has a chemotactic effect and can promote the migration and proliferation of dermal fibroblasts and synthesis of matrix macromolecules; its content is 1-20 μg/L. In a specific embodiment, content of the palmitoyl hexapeptide-12 is 3˜17 μg/L.

The laminin-derived peptide has functions similar to laminin and can promote cell adhesion and proliferation; its content is 10-100 μg/L. In a specific embodiment, content of the laminin-derived peptide is 30˜65 μg/L.

The above-mentioned tripeptide-1, tripeptide-2, hexapeptide-9, palmitoyl hexapeptide-12 and laminin-derived peptides gradually increase in performance as the content increases, but their performances are not greatly improved when the contents are beyond the upper limit of the application.

In the present disclosure, by introducing the above-mentioned polypeptides into the polypeptide composition, the polypeptides synergistically cooperates with the basal medium, so that the polypeptide composition promotes the rapid proliferation of mesenchymal stem cells.

As a serum-free medium for mesenchymal stem cells culture, the polypeptide composition also comprises a basic serum-free medium, which specifically includes: non-essential amino acids 1˜5 vol %; glutamine 1˜4 mmol/L; lipid mixture 1˜5 vol %; ITS (Insulin, Transferrin, Selenium) 1˜5 vol %; recombinant human serum albumin 1˜5 g/L; recombinant human epidermal growth factor 5˜25 μg/L; recombinant human fibronectin 5˜15 μg/L; L-glutathione peptide 1˜5 mg/L; β-mercaptoethanol 1˜5 mg/L; and α-MEM basal medium 10.2 g/L. The above-mentioned basic serum-free medium is used for the cultivation of mesenchymal stem cells.

In a specific embodiment, the polypeptide composition includes: the content of the tripeptide-1 is 10 μg/L, the content of the tripeptide-2 is 10 μg/L, the content of the hexapeptide-9 is 10 μg/L, the content of the palmitoyl hexapeptide is 10 μg/L, the content of the laminin-derived peptide is 60 μg/L, the content of the non-essential amino acid is 1 vol %, the content of the glutamine is 1˜4 mmol/L, the content of the lipid mixture is 1 vol %, the content of the ITS is 1 vol %, the content of the recombinant human serum albumin is 2.5 g/L, the content of the recombinant human epidermal growth factor is 20 μg/L, the content of the recombinant human fibronectin is 10 μg/L, the content of the L-glutathione is 2 mg/L, and the content of the β-mercaptoethanol is 2 mg/L.

The polypeptide composition described in the present disclosure can be prepared according to a method well known to those skilled in the art. After the components are mixed, the mixture is filtered and sterilized by passing the filter membrane, and then added to the α-MEM basal medium and mixed well. After that, the serum-free medium for promoting the proliferation of umbilical cord derived mesenchymal stem cells (UC-MSCs) was obtained.

The mesenchymal stem cell-promoting polypeptide composition provided by the present disclosure has definite chemical components, no animal origin and no serum. It realizes the rapid proliferation of UC-MSCs, solves the problem of insufficient cell numbers while maintaining the biological characteristics and immunity phenotypic stability of mesenchymal stem cells.

In order to further understand the present disclosure, the polypeptide composition for promoting the proliferation of mesenchymal stem cells provided by the present disclosure will be described in detail in conjunction with examples below. The protection scope of the present disclosure is not limited by the following examples.

The components and reagents used in the following examples are all commercial products. For example, α-MEM basal medium (Cat. No. 41060037) and non-essential amino acid solution (Cat. No. 11140-050) were purchased from Gibco; other components can be purchased from Sigma, MP, Gibco or other companies.

### EXAMPLE 1

### Serum-Free Medium Preparation

1) Based on α-MEM basal medium, a serum-free medium was prepared according to the following formula:

| | | |
|---|---|---|
| non-essential amino acids | 1 | vol % |
| glutamine | 1~4 | mmol/L |
| lipid mixture | 1 | vol % |
| ITS | 1 | vol % |
| recombinant human serum albumin | 2.5 | g/L |
| recombinant human epidermal growth factor | 20 | μg/L |
| recombinant human fibronectin: | 10 | μg/L |
| L-glutathione | 2 | mg/L |
| β-mercaptoethanol | 2 | mg/L |
| tripeptide-1 | 10 | μg/L |
| tripeptide-2 | 10 | μg/L |
| hexapeptide-9 | 10 | μg/L |
| palmitoyl hexapeptide-12 | 10 | μg/L |
| laminin-derived peptide | 50 | μg/L |
| α-MEM basal medium | 10.2 | g/L |

The above-mentioned components were dissolved in water at room temperature and fully dissolved to obtain the polypeptide composition of the present disclosure, namely experiment group 2. The composition was filtered and sterilized through a 0.22 μm filter membrane to promote the proliferation of mesenchymal stem cells.

2) Based on α-MEM basal medium, a serum-free medium was prepared according to the following formula:

| | | |
|---|---|---|
| non-essential amino acids | 0.01 | vol % |
| glutamine | 1~4 | mmol/L |
| lipid mixture | 1 | vol % |
| ITS | 1 | vol % |
| recombinant human serum albumin | 2.5 | g/L |
| recombinant human epidermal growth factor | 20 | μg/L |
| recombinant human fibronectin: | 10 | μg/L |
| L-glutathione | 2 | mg/L |
| β-mercaptoethanol | 2 | mg/L |
| tripeptide-1 | 20 | μg/L |
| tripeptide-2 | 20 | μg/L |
| hexapeptide-9 | 20 | μg/L |
| palmitoyl hexapeptide-12 | 20 | μg/L |
| laminin-derived peptide | 100 | μg/L |
| α-MEM basal medium | 10.2 | g/L |

The above-mentioned components were dissolved in water at room temperature and fully dissolved to obtain the polypeptide composition of the present disclosure, namely experiment group 3. The composition was filtered through a 0.22 μm filter for sterilization and then used for promoting the proliferation of mesenchymal stem cells.

3) Based on α-MEM basal medium, prepare basic serum-free medium according to the following formula:

| | | |
|---|---|---|
| non-essential amino acids | 1 | vol % |
| glutamine | 1~4 | mmol/L |
| lipid mixture | 1 | vol % |
| ITS | 1 | vol % |
| recombinant human serum albumin | 2.5 | g/L |
| recombinant human epidermal growth factor | 20 | μg/L |
| recombinant human fibronectin: | 10 | μg/L |
| L-glutathione | 2 | mg/L |
| β-mercaptoethanol | 2 | mg/L |
| α-MEM basal medium | 10.2 | g/L |

The above-mentioned components were dissolved in water at room temperature and fully dissolved to obtain the polypeptide composition of the present disclosure, namely experiment group 1. The composition was filtered and sterilized through a 0.22 μm filter membrane, and then used to promote the proliferation of mesenchymal stem cells.

4) Based on α-MEM basal medium, a serum-free medium was according to the following formula:

| | | |
|---|---|---|
| non-essential amino acids | 1 | vol % |
| glutamine | 1~4 | mmol/L |
| lipid mixture | 1 | vol % |
| ITS | 1 | vol % |
| recombinant human serum albumin | 2.5 | g/L |
| recombinant human epidermal growth factor | 20 | μg/L |
| recombinant human fibronectin: | 10 | μg/L |
| L-glutathione | 2 | mg/L |
| β-mercaptoethanol | 2 | mg/L |
| tripeptide-1 | 10 | μg/L |
| tripeptide-2 | 10 | μg/L |
| α-MEM basal medium | 10.2 | g/L |

The above components were dissolved in water at room temperature and fully dissolved to obtain the polypeptide composition of the present disclosure, namely the control group 3. The composition was filtered and sterilized through a 0.22 μm filter membrane, and then used to promote the proliferation of mesenchymal stem cells.

5) Based on α-MEM basal medium, a serum-free medium was prepared according to the following formula:

| | | |
|---|---|---|
| non-essential amino acids | 1 | vol % |
| glutamine | 1~4 | mmol/L |
| lipid mixture | 1 | vol % |
| ITS | 1 | vol % |
| recombinant human serum albumin | 2.5 | g/L |
| recombinant human epidermal growth factor | 20 | μg/L |
| recombinant human fibronectin: | 10 | μg/L |
| L-glutathione | 2 | mg/L |
| β-mercaptoethanol | 2 | mg/L |
| hexapeptide-9 | 10 | μg/L |
| palmitoyl hexapeptide-12 | 10 | μg/L |
| laminin-derived peptide | 50 | μg/L |
| α-MEM basal medium | 10.2 | g/L |

The above-mentioned components were dissolved in water and fully dissolved at room temperature to obtain the polypeptide composition of the present disclosure, namely the control group 4. The composition was filtered through a 0.22 μm filter membrane to be sterilized, and then used for promoting the proliferation of mesenchymal stem cells.

Experiment group 1, experiment group 2, experiment group 3, control group 3, and control group 4 were set using the medium prepared above, respectively. α-MEM complete medium containing 10% FBS was set as control group 1, STEMPRO® hMSC SFM (GIBCO) was set as the control group 2. The following experiments were performed.

### EXAMPLE 2

### Morphology Observation and Activity Detection of UC-MSCs

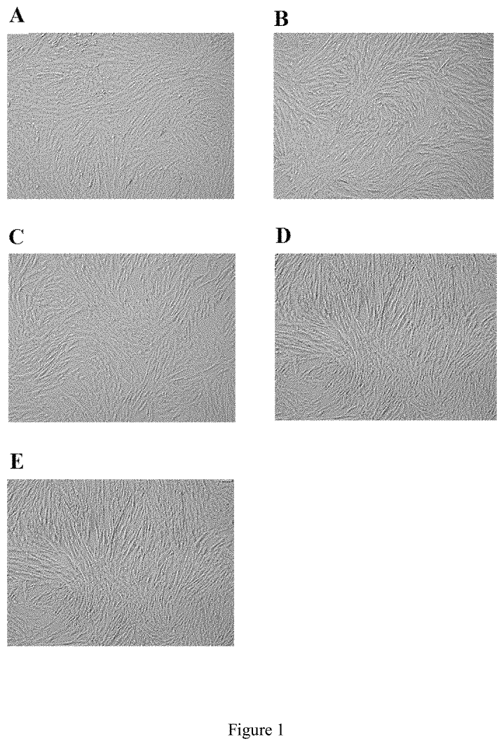

The P3 generation of UC-MSCs were selected to carry out the experiment. UC-MSCs were inoculated into T25 culture flasks at a density of 1×10⁴/cm², with 3 replicates in each group. The cells were cultured in a 5% CO₂ incubator at 37° C. for 48 hours and the images of the UC-MSCs were collected, the results were shown in FIG. 1. FIG. 1A showed the morphology of UC-MSCs in control group 1, FIG. 1B showed the morphology of UC-MSCs in control group 2, FIG. 1C showed the morphology of UC-MSCs in experiment group 1, FIG. 1D showed the morphological of UC-MSCs in experiment group 2, and FIG. 1E showed the morphology of UC-MSCs in experiment group 3.

It can be seen from FIG. 1 that the UC-MSCs of each group grew in a single layer, and most of the cells were long spindle-shaped with irregular morphology. The morphology of

UC-MSCs in the three experiment groups was more similar to that of the control group 2.

After cultured for 72 hours, the UC-MSCs of each group were digested with 0.25% trypsin solution, and a cell counter (Countstar) was used to count the number of cells and cell viability in each group was calculated. The results are shown in Table 1.

TABLE 1
Detection results of UC-MSCs in each group

| Experiment Group | Cell Viability (%) |
|---|---|
| Control Group 1 | 91.75 ± 0.11 |
| Control Group 2 | 93.20 ± 0.25 |
| Control Group 3 | 90.05 ± 0.34 |
| Control Group 4 | 91.86 ± 0.12 |
| Experiment Group 1 | 91.25 ± 0.35 |
| Experiment Group 2 | 98.66 ± 0.21 |
| Experiment Group 3 | 97.44 ± 0.14 |

The cell viabilities of experiment group 2 and experiment group 3 were compared with the control group 1, the control group 3, and the control group 4 respectively, and there were significant differences (p<0.05).

It can be seen from Table 1 that the UC-MSCs viability of the experiment group is higher than that of the control group 1 and the control group 2, and the UC-MSCs viability of the experiment group 2 and the experiment group 3 is higher than that of the experiment group 1.

### EXAMPLE 3

### Detection of UC-MSCs Proliferation Rate

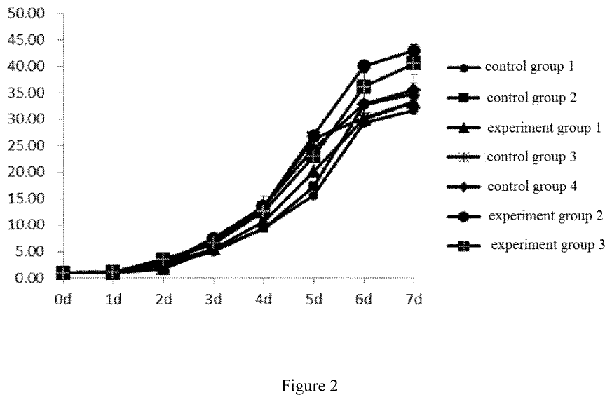

The P3 generation of UC-MSCs were selected to carry out the experiment. UC-MSCs were inoculated in a 24-well plate at a density of 1×10⁴ cells/ml and cultured in a 5% CO₂ incubator at 37° C. Cells were collected daily for cell count, and each time 3 wells were collected randomly and calculated, for 7 consecutive days. The cell growth curve was plotted and the results were shown in Table 2 and FIG. 2.

TABLE 2
7-day cell counting results of UC-MSCs in each group

| | Counts (×10\hat{ }4) | | | | | | | |
|---|---|---|---|---|---|---|---|---|
| Experiment Group | 0 d | 1 d | 2 d | 3 d | 4 d | 5 d | 6 d | 7 d |
| Control Group 1 | 1.00 ± 0.00 | 1.15 ± 0.09 | 2.30 ± 0.46 | 5.00 ± 0.38 | 9.45 ± 0.40 | 15.60 ± 1.08 | 29.25 ± 4.48 | 31.67 ± 2.23 |
| Control Group 2 | 1.00 ± 0.00 | 0.95 ± 0.23 | 3.00 ± 0.26 | 5.40 ± 0.44 | 9.40 ± 0.46 | 17.25 ± 0.40 | 32.80 ± 0.26 | 34.83 ± 1.99 |
| Control Group 3 | 1.00 ± 0.00 | 1.07 ± 0.05 | 2.44 ± 0.23 | 5.06 ± 0.19 | 13.28 ± 1.75 | 26.34 ± 2.61 | 30.16 ± 1.14 | 33.35 ± 2.09 |
| Control Group 4 | 1.00 ± 0.00 | 1.08 ± 0.02 | 3.06 ± 0.26 | 5.02 ± 0.28 | 13.62 ± 1.30 | 24.38 ± 1.80 | 32.85 ± 3.26 | 35.55 ± 2.70 |
| Experiment Group 1 | 1.00 ± 0.00 | 1.00 ± 0.17 | 1.80 ± 0.53 | 5.53 ± 0.56 | 9.70 ± 0.38 | 16.60 ± 2.42 | 30.05 ± 1.15 | 33.20 ± 0.71 |
| Experiment Group 2 | 1.00 ± 0.00 | 1.00 ± 0.09 | 2.70 ± 0.30 | 7.50 ± 0.40 | 13.55 ± 0.88 | 26.88 ± 0.75 | 40.05 ± 1.05 | 43.02 ± 1.05 |
| Experiment Group 3 | 1.00 ± 0.00 | 1.08 ± 0.11 | 3.50 ± 0.89 | 6.52 ± 1.19 | 12.62 ± 1.84 | 22.92 ± 1.86 | 36.09 ± 2.65 | 40.54 ± 2.90 |

Cell mass on day 7 of each group was analyzed for significance. The experiment group 2 and the experiment group 3 were compared with the control group 1, the control group 3 and the control group 4, respectively, and there were significant differences (p<0.05).

From the results in Table 2 and FIG. 2, compared with the experiment group 1 and the control groups, the UC-MSCs cultured by the polypeptide composition of the present disclosure have higher proliferation activity.

According to the doubling time calculation formula: DT=t*[lg2/(lgNt−lgNo)], wherein t is culture time; No is the number of cells recorded for the first time; Nt is the number of cells after t passages. The results are shown in Table 3.

TABLE 3
Doubling time of UC-MSCs in each group

| Experiment | Control | Control | Control | Control | Experiment | Experiment | Experiment |
|---|---|---|---|---|---|---|---|
| Group | Group 1 | Group 2 | Group 3 | Group 4 | Group 1 | Group 2 | Group 3 |
| Doubling | 32.12 ± 0.15 | 31.01 ± 0.21 | 32.22 ± 0.30 | 32.23 ± 0.32 | 29.18 ± 0.24 | 24.77 ± 0.17 | 26.62 ± 0.33 |
| Time (H) | | | | | | | |

The experiment group 2 and the experiment group 3 were compared with the control group 1, the control group 3, and the control group 4 respectively, and there were significant differences (p<0.05).

It can be seen from Table 3 that the doubling time of UC-MSCs in experiment group 2 and experiment group 3 is less than that in experiment group 1 and the four control groups, indicating that the proliferation rate of UC-MSCs cultured in the polypeptide composition of the present disclosure is higher.

### EXAMPLE 4

### UC-MSCs Surface Marker Detection

The P3 generation of UC-MSCs were selected to carry out the experiment. UC-MSCs were inoculated into T25 culture flasks at a density of 1×10⁴/cm² and cultured in a 5% CO₂ incubator at 37° C. After 3 days, the UC-MSCs of each group were collected by digestion with 0.25% trypsin solution, and the expression of surface markers such as CD105, CD73, CD90, CD34, CD45, HLA-DR, etc. were detected by flow cytometry. The results are shown in Table 4.

TABLE 4
Surface marker detection results of UC-MSCs in each group

| Experiment Group | CD105 | CD90 | CD73 | CD34 | CD45 | HLA-DR |
|---|---|---|---|---|---|---|
| Control Group 1 | 100.0% | 98.50% | 96.35% | 0.02% | 0.15% | 0.01% |
| Control Group 2 | 99.96% | 100.0% | 95.24% | 0.01% | 0.00% | 0.03% |
| Control Group 3 | 100.0% | 98.35% | 96.76% | 0.00% | 0.00% | 0.02% |
| Control Group 4 | 97.95% | 99.25% | 96.33% | 0.04% | 0.00% | 0.02% |
| Experiment Group 1 | 99.45% | 100.0% | 98.10% | 0.00% | 0.01% | 0.02% |
| Experiment Group 2 | 100.0% | 97.46% | 99.80% | 0.01% | 0.00% | 0.01% |
| Experiment Group 3 | 100.0% | 99.25% | 100.0% | 0.02% | 0.00% | 0.01% |

It can be seen from Table 4 that the surface markers CD105, CD73, and CD90 of UC-MSCs are expressed in each experiment groups and control groups, while CD34, CD45, and HLA-DR are not expressed, and there is no significant difference between the groups, indicating that the polypeptide composition of present disclosure does not affect the expression of UC-MSCs surface markers during culturing.

### EXAMPLE 5

### Detection of Multi-Directional Differentiation Potential of UC-MSCs

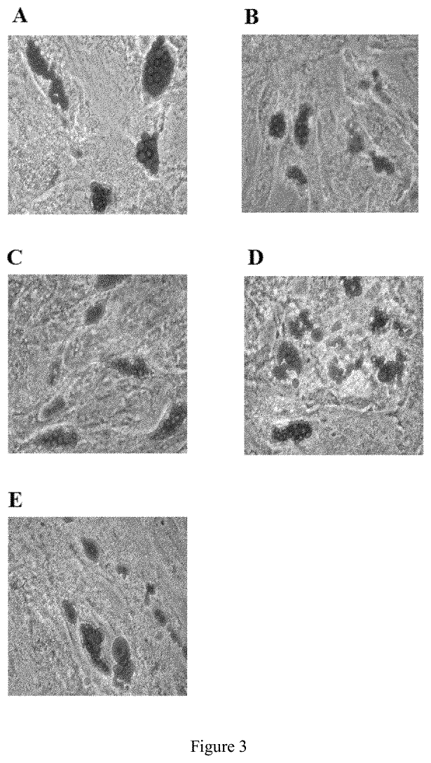

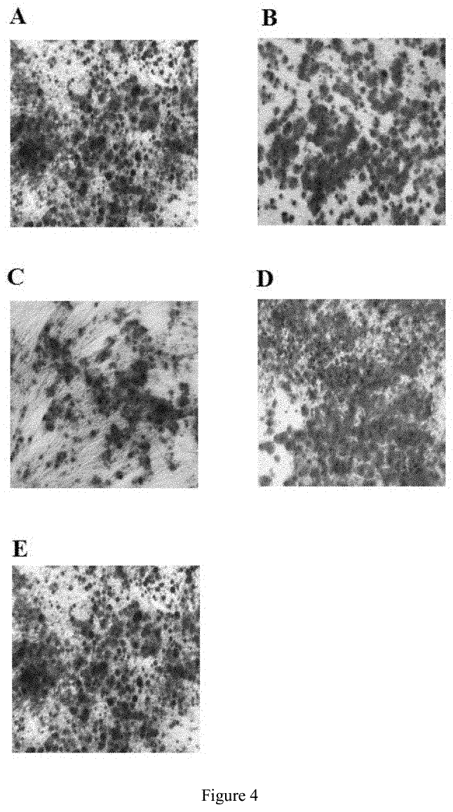

The P3 generation UC-MSCs were selected for the experiment. The UC-MSCs of the experiment groups 1˜3, the control group 1 and the control group 2 were routinely cultured and passaged to the P5 generation, and then inoculated in a 6-well plate at a density of 1×10⁵/ml. The cells were incubated at 37° C. in a 5% CO₂ incubator. When the confluence of UC-MSCs in each group reached more than 80%, control wells and induction wells were set respectively and osteogenic and adipogenic differentiation of UC-MSCs were induced. After 14 days, the cells in the adipogenic differentiation experiment group were stained with Oil Red O. And after 21 days, the cells in the osteogenic differentiation experiment group were stained with Alizarin Red. The results are shown in FIG. 3 and FIG. 4.

FIG. 3A is the adipogenic differentiation result of UC-MSCs of control group 1, FIG. 3B is the adipogenic differentiation result of UC-MSCs of control group 2, and FIG. 3C is the adipogenic differentiation result of UC-MSCs of experiment group 1, FIG. 3D is the adipogenic differentiation result of UC-MSCs of experiment group 2, FIG. 3E is the adipogenic differentiation result of UC-MSCs of experiment group 3.

FIG. 4A is an image of the osteogenic differentiation result of UC-MSCs in control group 1, FIG. 4B is an image of the osteogenic differentiation result of UC-MSCs in control group 2, FIG. 4C is an image of the osteogenic differentiation of UC-MSCs in experiment group 1, FIG. 4D is an image of the osteogenic differentiation result of UC-MSCs in experiment group 2, and FIG. 4E is an image of the osteogenic differentiation result of UC-MSCs in experiment group 3. The experiment results show that the culture of UC-MSCs supplemented with the polypeptide combination of the present disclosure will not affect their adipogenic and osteogenic differentiation potential and maintain their stemness.

The description of the above embodiments is only used to help the understanding of the method and the core idea of the present disclosure. It should be pointed out that for those of ordinary skilled in the art, several improvements and modifications can be made to the present disclosure without departing from the principle of the present disclosure, and these improvements and modifications also fall within the protection scope of the claims of the present disclosure.

The foregoing description of the disclosed embodiments enables those skilled in the art to implement or employ the present disclosure. Various modifications to these embodiments will be obvious to those skilled in the art, and the general principles defined herein can be implemented in other embodiments without departing from the spirit or scope of the present disclosure. Therefore, the present disclosure will not be limited to the embodiments shown in this text, but would conform to the widest scope consistent with the principles and novel features disclosed herein.

What is claimed is:

1. A serum-free polypeptide composition for promoting proliferation of mesenchymal stem cells, comprising:

| | | |
|---|---|---|
| tripeptide-1 | 1~20 | μg/L; |
| tripeptide-2 | 1~20 | μg/L; |
| hexapeptide-9 | 1~20 | μg/L; |
| palmitoyl hexapeptide-12 | 1~20 | μg/L; |
| laminin-derived peptide | 10~100 | μg/L; |
| non-essential amino acids | 1~5 | vol %; |
| glutamine | 1~4 | mmol/L; |
| lipid mixture | 1~5 | vol %; |
| ITS (Insulin, Transferrin, Selenium) | 1~5 | vol %; |
| recombinant human serum albumin | 1~5 | g/L; |
| recombinant human epidermal growth factor | 5~25 | μg/L; |
| recombinant human fibronectin | 5~15 | μg/L; |
| L-glutathione | 1~5 | mg/L; |
| β-mercaptoethanol | 1~5 | mg/L; and |
| α-MEM basal medium | 10.2 | g/L. |

2. The serum-free polypeptide composition according to claim 1, wherein content of the tripeptide-1 is 3-18 μg/L.

3. The serum-free polypeptide composition of claim 1, wherein content of the tripeptide-2 is 3-18 μg/L.

4. The serum-free polypeptide composition of claim 1, wherein content of the hexapeptide-9 is 4-18 μg/L.

5. The serum-free polypeptide composition of claim 1, wherein content of the palmitoyl hexapeptide-12 is 3-17 μg/L.

6. The serum-free polypeptide composition of claim 1, wherein content of the laminin-derived peptide is 30-65 μg/L.

7. The serum-free polypeptide composition of claim 1, wherein content of the tripeptide-1 is 10 μg/L, content of the tripeptide-2 is 10 μg/L, content of the hexapeptide-9 is 10 μg/L, content of the palmitoyl hexapeptide is 10 μg/L, and content of the laminin-derived peptide is 60 μg/L.

8. The serum-free polypeptide composition according to claim 7, wherein content of the non-essential amino acid is 1 vol %, content of the glutamine is 1 to 4 mmol/L, content of the lipid mixture is 1 vol %, content of the ITS is 1 vol %, content of the recombinant human serum albumin is 2.5 g/L, content of the recombinant human epidermal growth factor is 20 μg/L, content of the recombinant human fibronectin is 10 μg/L, content of the L-glutathione is 2 mg/L, and content of the β-mercaptoethanol is 2 mg/L.
